(19) United States
(12) Patent Application Publication
(10) Pub. No.: US 2017/0119248 A1
(43) Pub. Date: May 4, 2017

(54) MONITORING DROWSINESS

(71) Applicant: SDIP HOLDINGS PTY LTD, Richmond, Victoria (AU)

(72) Inventors: Trefor MORGAN, Brunswick (AU); Scott COLES, Waverton (AU); Andrew TUCKER, Montmorency (AU)

(73) Assignee: SDIP HOLDINGS PTY LTD, Richmond, Victoria (AU)

(21) Appl. No.: 15/318,417

(22) PCT Filed: Jun. 19, 2015
(86) PCT No.: PCT/AU2015/000359

§ 371 Date: Dec. 13, 2016

(30) Foreign Application Priority Data
Jun. 20, 2014 (AU) 2014902364

(51) Int. Cl. A61B 3/113; G06K 9/00; A61B 5/16; A61B 5/00; A61B 3/14; A61B 5/11

(52) U.S. Cl. CPC A61B 3/113; A61B 3/14; A61B 5/1128; A61B 5/1114; A61B 5/16; A61B 5/1103; A61B 5/7278; G06K 9/00604

(57) ABSTRACT

Lower sampling rates have been found to provide sufficient data for use in the method of U.S. Pat. Nos. 7,071,831, 7,616,125 and 7,791,491. The method of determining drowsiness includes the steps of receiving eye movement data collected at sampling rates as low as 20 Hz. The data is preferentially interpolated to provide a data set at a higher sampling rate in the order of 500 Hz. For each data point, values of amplitude and velocity of eye movement and whether the measures relate to eyelid opening or closing are derived. An algorithm is then used to obtain values of the amplitude to velocity ratios of eyelid opening and closing and using these values in an algorithm for providing a measure of drowsiness. With this method eyelid and eye movement may be monitored using any suitable technology or sensor including video or digital camera technology to identify and measure the appropriate ocular movements.

This invention relates to methods of measuring drowsiness and in particular of using eyelid movement data from any source that has low sampling rates.

### BACKGROUND TO THE INVENTION

U.S. Pat. Nos. 7,071,831, 7,616,125 and 7,791,491 relate to a method and algorithm for predicting the onset of potentially fatal drowsiness. The method in practice uses spectacle mounted IR sensors to sense eye and eyelid movement and measure amplitude and velocity of these movements for processing in an algorithm that provides a measure that is applicable to a scale of drowsiness. Other sensor systems such as cameras could be used to collect this data but have not been used as their sampling rates are low. The attraction of a camera based system is that spectacle need not be worn to collect the data and is thus less intrusive. U.S. Pat. No. 7,043,056 to a camera based method of determining a head pose measurement. U.S. Pat. No. 7,460,693 to a camera based method of locating a face within an input image including eye locations. U.S. Pat. No. 7,653,213 to a face tracking system utilising a Kalman filter and deriving a Jacobian. U.S. Pat. No. 8,165,347 to a face tracking system which includes a determination if glasses are being worn.

There are many patents to eye tracking methods. Some of these are for use in anticipating a screen users requirements as part of a computer or mobile phone system. Other patents are concerned with identification by iris examination and an example is U.S. Pat. No. 8,064,647.

Some patents are more focussed on analysing eye movements such as U.S. Pat. No. 7,809,160 for eye tracking without camera calibration.

Camera based systems have been proposed for detecting drowsiness but their ability to provide reliable early predictions in a high percentage of cases is questionable.

It is an object of this invention to provide a method and apparatus that enables other sensor inputs of eye movement data.

### BRIEF DESCRIPTION OF THE INVENTION

To this end the present invention provides a method of determining drowsiness which includes the steps of

receiving eye movement data collected at sampling rate greater than 20 Hz but less than 250 Hz

interpolating the data to provide a data set sampling rates greater than 250 Hz for each data point deriving values of amplitude and velocity of eye movement and whether the measures relate to eyelid opening or closing

using an algorithm to obtain values of the amplitude to velocity ratios of eyelid opening and closing and using these values in an algorithm for providing a measure of drowsiness.

This invention is predicated on the unexpected discovery that lower sampling rates can provide sufficient data for use in the method of U.S. Pat. Nos. 7,071,831, 7,616,125 and 7,791,491. The system of those patents uses a sample rate of 500 Hz to gather amplitude measurements for eyelid movements. In this invention the sampling rates for various type of sensor devices may be 50 Hz, 100 Hz, 200 Hz. Preferably the upper limit for the sample rate of the eye movement data source is 200 Hz.

An alternate embodiment of the invention avoids the need to adjust the sample rate of the eye movement data source (such as that obtained from a video camera). Modifications to the methods of collation and calculation of eye movement amplitude, velocity and durations used in the JDS algorithm may be adjusted to account for low sampling rates.

Thus in another embodiment this invention provides a method of determining drowsiness which includes the steps of

receiving eye movement data collected at sampling rate greater than 20 Hz but less than 250 Hz

collating and calculating eye movement amplitude, velocity and duration;

for each data point, deriving values of amplitude and velocity of eye movement and whether the measures relate to eyelid opening or closing;

using an algorithm to obtain values of the amplitude to velocity ratios of eyelid opening and closing and

using these values in an algorithm for providing a measure of drowsiness.

The amplitude to velocity ratio for eyelid opening and closing are used as the main measure of drowsiness onset In the John Drowsiness Scale (JDS). The ratio of the amplitude of to the maximum velocity (AVR) for both closing and opening phases during blinks increases with drowsiness and can be used to predict lapses in vigilance. The AVR for eyelid closure and reopening are different for the same amplitude. Generally eyelids close more quickly than they reopen and the two velocities are only moderately correlated. Sleep deprivation, restriction or other reasons for drowsiness increases AVR for both closing and reopening.

Consequently the duration of these movements increase with drowsiness. It has been found that the ratio of opening and closing velocities with their respective amplitudes are a major indicator of drowsiness. The ratio of the amplitude of opening and closing movements relative to the maximum velocity (AVR) of these movements has the dimension of time and is relatively constant with alert subjects but increases progressively with drowsiness and does not require calibration.

The values calculated for the purposes of comparison need to be collated over a predetermined period of time and expressed in an appropriate way. These calculations are preferentially expressed as averages, standard deviations, percentiles or counts. The eyelid parameters measured and the values selected for calculation can be determined by conducting trials and may be any suitable combination of parameters. Preferably the velocity to amplitude ratios are calculated for each detected movement and then expressed as averages and standard deviations over a predetermined interval. Other parameters such as duration of opening, closure and closing may also be collated and included in the final calculated value. Eye movements such as saccades may be used as additional parameters if they are available from the data source. The various parameters are preferably weighted in reaching the final calculation. This final calculation becomes an index of drowsiness with a low value indicating alertness and higher values indicating increasing levels of drowsiness.

Eyelid and eye movement may be monitored using any suitable technology or sensors including video or digital camera technology to identify and measure the appropriate eye movements.

### DETAILED DESCRIPTION OF THE INVENTION

A preferred embodiment of the invention will now be described with reference the drawings in which:

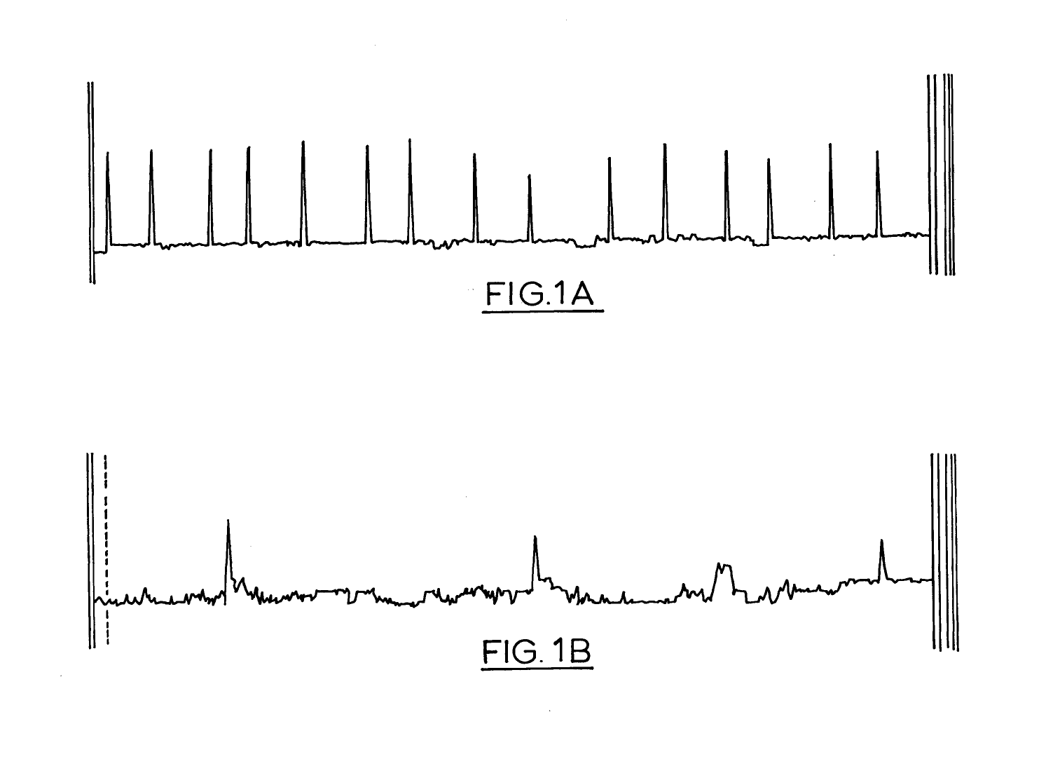

FIG. 1A is a good quality eyelid aperture data signal derived from video showing a 30 second period of blinks;

FIG. 1B is a noisy eyelid aperture data signal derived from video showing a 24 second period of noise between blinks;

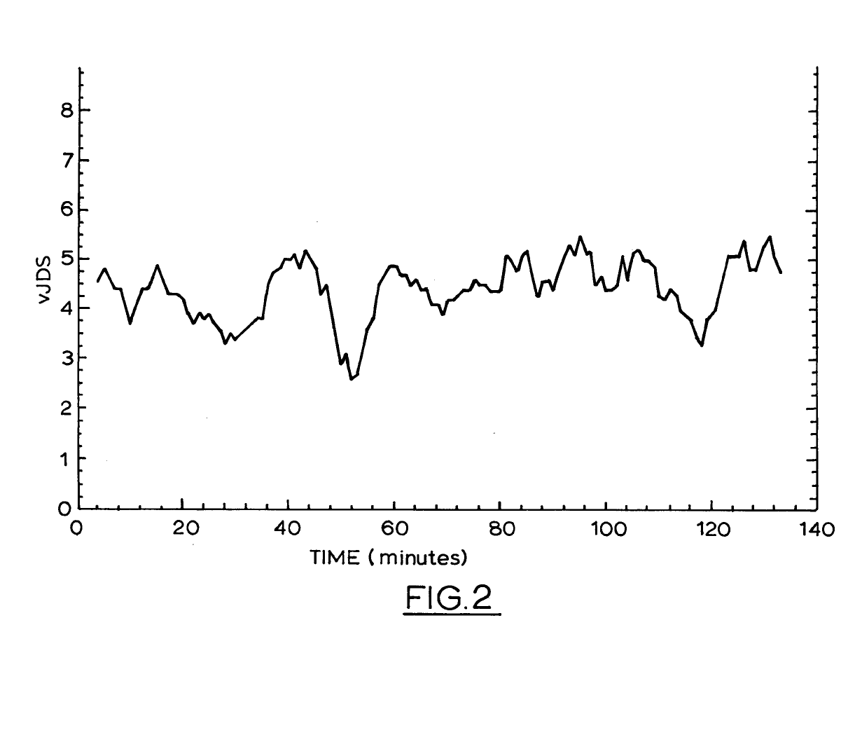

FIG. 2 illustrates a graph of Johns drowsiness score derived from video based signals over 120 minutes;

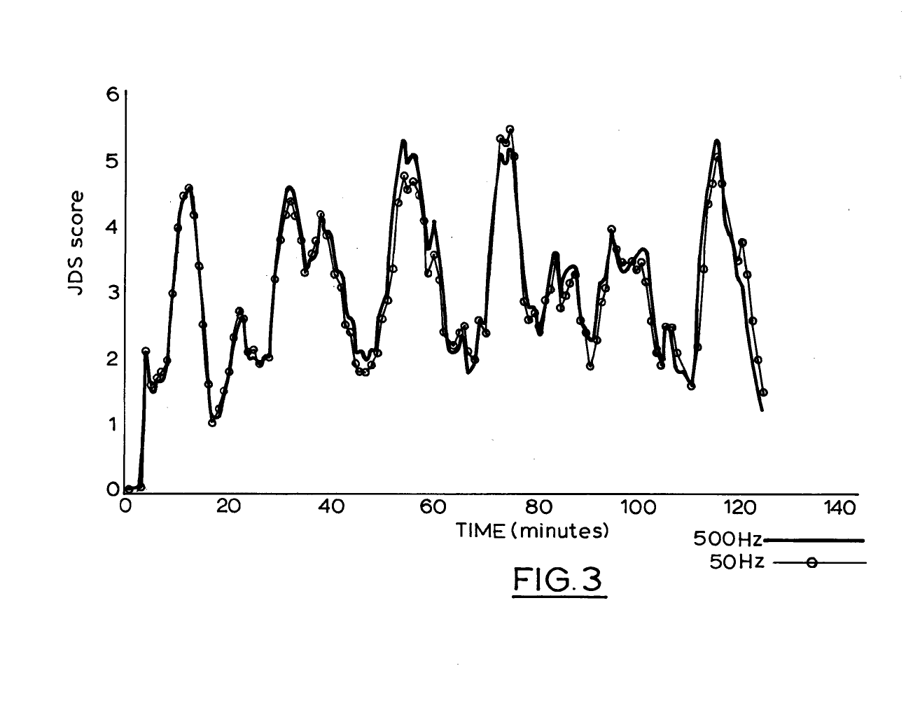

FIG. 3 illustrates JDS score for 500 Hz vs 50 Hz; 50 Hz to reflect the applicability of JDS for lower sampling rates;

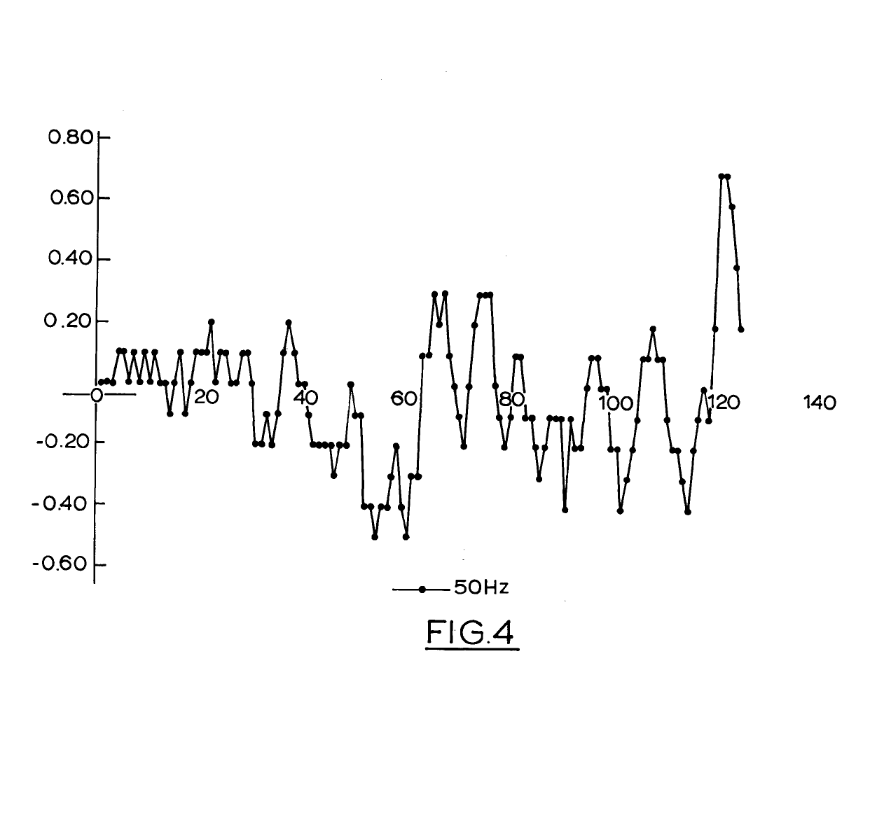

FIG. 4 illustrates minimal error of 50 Hz signal against the original 500 Hz signal.

A data set of eyelid movements obtained using a camera-based system was obtained using a third party program which supplied the data as a CSV output file with time and eye opening (aperture) value for each video sample.

The data was then upscaled to 500 Hz and the amplitude rescaled.

Initial analysis of the data is shown in FIG. 1A which is a good quality signal and FIG. 1B illustrates that blinks can be identified in a noisy signal. After converting and interpolating the video camera output, analysis by the JDS algorithms show in FIG. 2 a JDS score over a period of two hours.

To investigate the feasibility of using a low sampling rate as in data from a camera, a sample data set with a sampling rate of 500 Hz was adjusted to a sample rate of 50 Hz. This was then converted back up to a sample rate of 500 Hz by interpolating between the data points of the 50 Hz sample. Linear interpolation is the preferred method, but other methods of interpolation such as sample and hold, polynomial interpolation or spline interpolation can be used.

FIG. 3 illustrates the difference in the JDS scores over time derived from the original 500 Hz data and that derived from the 50 Hz data set. FIG. 4 illustrates the error of the JDS scores derived from the 50 Hz data set against the original 500 Hz data set. This demonstrates that the use of a lower sampling frequency provides reliable scores.

An alternate embodiment of the invention is possible without the need to adjust the sample rate of the eye movement data source (such as that obtained from a video camera). Modifications to the methods of collation and calculation of eye movement amplitude, velocity and durations may be adjusted to account for alternate sampling rates.

Those skilled in the art will realize that this invention improves the functionality of the applicants drowsiness algorithm by making it able to accept data collected by any means at lower sampling frequencies.

Those skilled in the art will also realize that this invention may be implemented in embodiments other than those described without departing from the core teachings thereof.

1. A method of determining drowsiness which includes the steps of receiving eye movement data collected at sampling rate greater than 20 Hz but less than 250 Hz;
collating and calculating eye movement amplitude, velocity and duration; for each data point deriving values of amplitude and velocity of eye movement and whether the measures relate to eyelid opening or closing;
using an algorithm to obtain values of the amplitude to velocity ratios of eyelid opening and closing; and
using these values in an algorithm for providing a measure of drowsiness.

2. A method of determining drowsiness which includes the steps of receiving eye movement data collected at sampling rate greater than 20 Hz but less than 250 Hz;
interpolating the data to provide a data set sampling rates greater than 250 Hz for each data point deriving values of amplitude and velocity of eye movement and whether the measures relate to eyelid opening or closing;
using an algorithm to obtain values of the amplitude to velocity ratios of eyelid opening and closing and using these values in an algorithm for providing a measure of drowsiness.

3. The method as claimed in claim 1, in which the eye movement data is collected by a camera.

4. The method as claimed in claim 1, in which the eye movement data is eyelid aperture data.

5. The method as claimed in claim 2, in which the eye movement data is collected by a camera.

6. The method as claimed in claim 2, in which the eye movement data is eyelid aperture data.
